(19) United States
(12) Patent Application Publication
(10) Pub. No.: US 2021/0389254 A1
(43) Pub. Date: Dec. 16, 2021

(54) METHOD FOR MEASURING CONCENTRATION OF POLYELECTROLYTE AND PHOSPHONATE BLENDS

(71) Applicant: Kemira Oyj, Helsinki (FI)

(72) Inventors: Salla PUUPPONEN, Espoo (FI); Sari KRAPU, Espoo (FI)

(21) Appl. No.: 17/281,804

(22) PCT Filed: Sep. 27, 2019
(86) PCT No.: PCT/FI2019/050694

§ 371 Date: Mar. 31, 2021

(30) Foreign Application Priority Data
Oct. 1, 2018 (FI) 20185816

(51) Int. Cl. G01N 21/77; G01N 21/64; G01N 1/28; G01N 31/22

(52) U.S. Cl. CPC G01N 21/77; G01N 21/6408; G01N 21/6428; G01N 2021/7786; G01N 31/22; G01N 2021/6439; G01N 1/28

(57) ABSTRACT

The present invention relates to utilization of lanthanide time resolved fluorescence for determining total concentration of polyelectrolyte or phosphonate in a sample. In the method sample comprising two or more polyelectrolytes and/or phosphonates is acidified followed by admixing the acidified sample with a reagent comprising a lanthanide(lll) ion. The polyelectrolyte or phosphonate in the sample are allowed to interact with the reagent comprising the lanthanide(lll) ion, followed by exciting the sample and detecting a signal deriving from the lanthanide(lll) ion, and determining the concentration of the polyelectrolyte or phosphonate in the sample by using the detected signal.

### FIELD OF INVENTION

The present invention relates to a method for determining concentration of polyelectrolyte and phosphonate blends in a sample with time-resolved fluorescence.

### BACKGROUND

Quantification of polyelectrolytes such as organic polymers is essential in many fields of industry, such as in water treatment, paper and oil industries. The organic polymers desired to be analyzed may be corrosion inhibitors, antiscalants or enhanced oil recovery (EOR) polymers.

Organic polymers can be measured in a rapid and easy manner by utilizing time-resolved fluorescence (TRF) of lanthanides(III). Europium(III) and terbium(III) are the most used lanthanide ions in TRF measurements, but also other lanthanides can be used. Organic polymers containing two or more chelating groups often increase the inherently weak TRF intensity of lanthanide(III) ions.

The chelating groups of the polymer may be e.g. carboxylates, sulfonates, carboxamides, phosphates, phosphonates or amines. The signal amplification depends on the polymer chemistry and concentration. Therefore, the signal amplification of polymer-lanthanide(III) chelates can be exploited for quantification of polymer samples, given that the chemistry (particularly that of chelating groups) of the sample does not substantially vary.

Often, polymer blends are used as products instead of single polymers. For instance, copolymer of sodium allyl sulfonate and maleate (SASMAC) scale inhibitors may be used together with polyacrylate type antiscalants as a blend in water treatment processes due to their different operation principles. If the fractions of the polymers in the blend change in use, reliable quantification has been challenging or even impossible, because the polymers give different TRF signals.

Therefore, there is need for a simple and reliable method for measuring total concentration of polyelectrolyte and phosphonate blends.

### SUMMARY OF INVENTION

An object of the present invention is to provide method for measuring total concentration of polyelectrolyte and phosphonate blends.

Another object of the present invention is to provide a simple and reliable method for measuring total concentration polyelectrolyte and phosphonate blends.

Chelation tendency and TRF signal amplification of polyelectrolyte or phosphonate-lanthanide mixtures depend often substantially on the charging (chelating groups) of the polymer.

In the present invention, the different TRF signals of distinct polyelectrolytes are standardized by suitable sample pre-treatment and/or modification of the measurement conditions.

It was found that the TRF signal intensity of lanthanide(III)-polyacrylic acid mixtures was substantially higher than those of lanthanide(III)-polyacrylates and lanthanide(III)-SASMAC mixtures.

It was also surprisingly found that the different signals of the polyelectrolyte and phosphonate could be standardized to the same level by acidification of the sample. Suitable acidification treatments change the TRF signal of the different polymers into the same level, enabling quantification of polyelectrolyte and phosphonate blends independent on the fractions of the polymers present in the sample.

By combining the total measurement of the present invention with suitable separation methods of the analytes or measurement methods specific for single polyelectrolyte or phosphonate in blend, the polyelectrolyte and phosphonate blend compositions can be completely solved.

In the method of the present invention aqueous sample solution is acidified to suitable pH range, such as pH less than 7. Preferably the pH of the solution is acidified to at most 6 such as 4-6.

In one embodiment, when sample matrix chemistry varies, the pH level can be standardized by using buffer in the acidification reaction. Preferably, the buffer is non-chelating, zwitterionic Good's buffer, such as HEPES or tris-bis propane. The buffer is selected so that it works in the modified pH range.

After standardization procedures, all the analytes (polyelectrolytes and phosphonates) give similar TRF signal. The signal of the blend can be converted into ppm range by comparing the signal to calibration curve. The calibration standards should be treated in the same manner as the analytes.

The polyelectrolytes contain two or more chelating groups selected from esters, ethers, hydroxyls, thiols, carboxylates, sulfonates, amides, phosphates, phosphonates and/or amines.

### BRIEF DESCRIPTION OF FIGURES

FIG. 1 illustrates time-resolved fluorescence signal of lanthanide(III)-polymer chelates.

FIG. 2 illustrates time-resolved fluorescence signal of Europium with 0-10 ppm polymeric scale inhibitors.

### DETAILED DESCRIPTION

The present invention provides a method for determining total concentration of polyelectrolyte and phosphonate blends in a sample. More particularly the present invention provides a method for determining total concentration of polyelectrolyte and phosphonate in a sample comprising two or more polyelectrolyte or phosphonate, the method comprising
optionally diluting and/or purifying the sample,
acidifying the sample to pH less than 7,
admixing the acidified sample with a reagent comprising lanthanide(III) ion,
allowing the organic polymers in the sample to interact with the reagent comprising the lanthanide(III) ion,
exciting the sample at an excitation wavelength and detecting a sample signal deriving from the lanthanide(III) ion at a signal wavelength by using time-resolved fluorescence, and
determining the total concentration of polyelectrolyte and phosphonate in the sample by using the detected sample signal.

The pH of the sample is preferably adjusted to less than 6 and more preferably to range 4-6.

In one embodiment the sample is acidified with an acid selected from a group consisting of HCl, HNO₃, H₂SO₄ or H₃PO₄, preferably HCl.

In a preferred embodiment additionally a buffer is admixed with the sample, preferably non-chelating buffer, more preferably zwitterionic Good's buffer, even more preferably HEPES or tris-bis propane.

In one embodiment concentration of the polyelectrolyte or phosphonate in the measurement mixture is in the range of 0.5-50 ppm, preferably 5-20 ppm.

In case the concentration of the polyelectrolyte and phosphonate in the sample is higher, the sample can be diluted.

In one embodiment concentration of the lanthanide(III) ion in the measurement mixture is in the range of 1-100 μM, preferably 1-20 μM.

By term “measurement mixture” is meant the admixture in the measurement.

The lanthanide(III) ion is selected from europium, terbium, samarium or dysprosium ions. The lanthanide (III) ion is preferably europium or terbium ions.

In a preferred embodiment the lanthanide(III) is a lanthanide(III) salt. The lanthanide(III) salt is selected from halogenides and oxyanions, such as nitrates, sulfates or carbonates, preferably from hydrated halogenides or nitrates, more preferably hydrated chloride.

The sample is optionally diluted to suitable aqueous solution e.g. deionized water or brine containing monovalent and/or divalent ions. Preferably, the dissolution brine does not contain any trivalent ions. Preferably the sample is an aqueous solution.

If the sample solution contains some interfering compounds such as trivalent metal cations or chelating agents that may affect TRF signal, suitable purification procedures may be applied prior to the dilution steps.

The sample is optionally purified by using a purification method selected from centrifugation, size exclusion chromatography, cleaning with solid-phase extraction (SPE) cartridges, dialysis techniques, extraction methods for removing hydrocarbons, filtration, microfiltration, ultrafiltration, nanofiltration, membrane centrifugation and any combinations thereof.

The analyte, polyelectrolyte or phosphonate, in the sample bears one or more groups that can hydrolyze and/or that the sample bears one or more groups that are capable of dissociating in aqueous solution to form either anion or cation groups. The analyte can be zwitterionic, i.e. contain both cationic and anionic groups. The charging of the group can depend on the environment pH (acidic or basic groups, such as carboxylic acids and amino groups). Therefore, the groups capable for dissociation can be neutral in certain pH (e.g. carboxylic acid group in acidic and amino groups in basic environment). The polyelectrolyte can be basic or acidic.

In one embodiment the polyelectrolyte contains one or more groups selected from, carboxylic acid/carboxylate, amide, phosphonate, amine, or any combination thereof.

In a preferred method the polyelectrolyte contain aromatic groups. The aromatic group(s) amplify the signal of lanthanide(III) ion.

In one embodiment the polyelectrolyte has molecular weight of at least 1000 g/mol.

In another embodiment the phosphonate has molecular weight of at least 100 g/mol.

To determine concentration of one polyelectrolyte or phosphonate in the blend said polyelectrolyte or phosphonate can be tagged.

In one embodiment at least one of the polyelectrolyte or phosphonate is tagged, preferably with fluorescent tag, such as sodium styrene sulfonate (NaSS), with different excitation and emission wavelengths as compared to the used lanthanide(III).

In said embodiment, after the determination of the total concentration of the polyelectrolyte or phosphonate polymers in the sample, the sample is
excited at excitation wavelength specific for the tag and detecting a sample signal deriving from the lanthanide(III) ion at a signal wavelength specific for the tag by using time-resolved fluorescence, and
determining the concentration of the at least one tagged polyelectrolyte or phosphonate in the sample by using the detected sample signal.

Unknown concentration of the polyelectrolyte or phosphonate in the sample is determined by comparing the sample signal to calibration curve. The calibration curve is obtained from TRF measurement of calibration standard samples with varying polyelectrolyte or phosphonate concentrations. Same dilution and or purification steps and measurement parameters have to be used for both the sample and calibration samples.

The lanthanide(III) ion is excited at excitation wavelength and measured at emission wavelength and detected by using time-resolved fluorescence (TRF). Any TRF reader can be employed. Excitation and emission wavelengths are selected so that the S/N is the best. Also the delay time can be optimized.

The excitation and emission wavelengths and the delay time are chosen based on the requirements of the lanthanide ion.

In an exemplary embodiment excitation wavelength and emission wavelength and delay time for Europium is 395 nm and 615 nm and 400 μs respectively.

The present invention further relates to use of the method of the present invention for determining total concentration of polyelectrolyte and/or phosphonate in a sample.

The sample can originate from water treatment, paper making processes, pharmaceutical industry, well drilling, mineral processing, enhanced oil recovery, an oilfield or an oil well or from an oil production process.

The present invention further relates a device comprising means for performing the method according to the present invention for determining total concentration of polyelectrolyte or phosphonate in a sample.

The examples are not intended to limit the scope of the invention but to describe embodiments of the invention.

### EXAMPLES

### Example 1. Standardization (Modification) of the TRF Signal of Polyacrylic Acids, Polyacrylates, SASMAC and AA-AMPS Copolymers or a Polymer Blend Thereof (According to the Invention)

The polymer or polymer blend is optionally diluted to suitable concentration range. Preferably, the active concentration of the sample and europium lanthanide are 0.5-50 ppm and μM, respectively. Optionally the polymer is diluted to suitable aqueous solution e.g. deionized water or brine containing monovalent and/or divalent ions. Preferably, the dissolution brine does not contain any trivalent ions. If the polymer solution contains some interfering compounds, suitable pretreatment procedures may be applied prior to the dilution steps. In the example, the polymers were diluted into a brine, which TDS varied between 20 000 and 40 000 ppm. The brines contain alkaline and earth alkaline metals as chlorides or bicarbonates.

After the optional dilution of the polymer, the solution is acidified to suitable pH range. Preferably the pH is under 6.

If the sample matrix chemistry varies, the pH level can be standardized also by using buffer in the reaction. Preferably, the buffer is non-chelating, zwitterionic Good's buffer, such as HEPES or tris-bis propane. The buffer is selected so that it works in the selected, modified pH range, e.g. in pH of 4-6.

After the standardization (modification) procedures, all the analytes give similar TRF signal. The signal of the blend can be converted into ppm range by comparing the signal to the calibration curve. The calibration standards should be treated in the same manner as the analytes.

### Example 2. Binary Mixture of Polyacrylic Acid and Fluorescence Tagged SASMAC (According to the Invention)

The binary mixture composition of polyacrylic acid and fluorescence tagged SASMAC can be examined by first using the modification method presented in Example 1. In the modification, the high signal of polyacrylic acid is reduced into the same level as that of SASMAC.

After total measurement, tagged SASMAC polymer can be measured with fluorescence method specific for the tag. For instance, sodium styrene sulfonate (NaSS) tagged SASMAC polymer can be quantified by fluorescence measurement of the product. Tag specific excitation and emission wavelengths are used in the measurement. In the case of NaSS tag, excitation and emission wavelengths of 225 nm and 285 nm can be used. The calibration standards of NaSS tagged SASMAC are measured similarly as the product and the fluorescence signal are converted into concentration of the polymer.

Polyacrylic acid concentration is obtained by subtracting the SASMAC polymer concentration from the total signal.

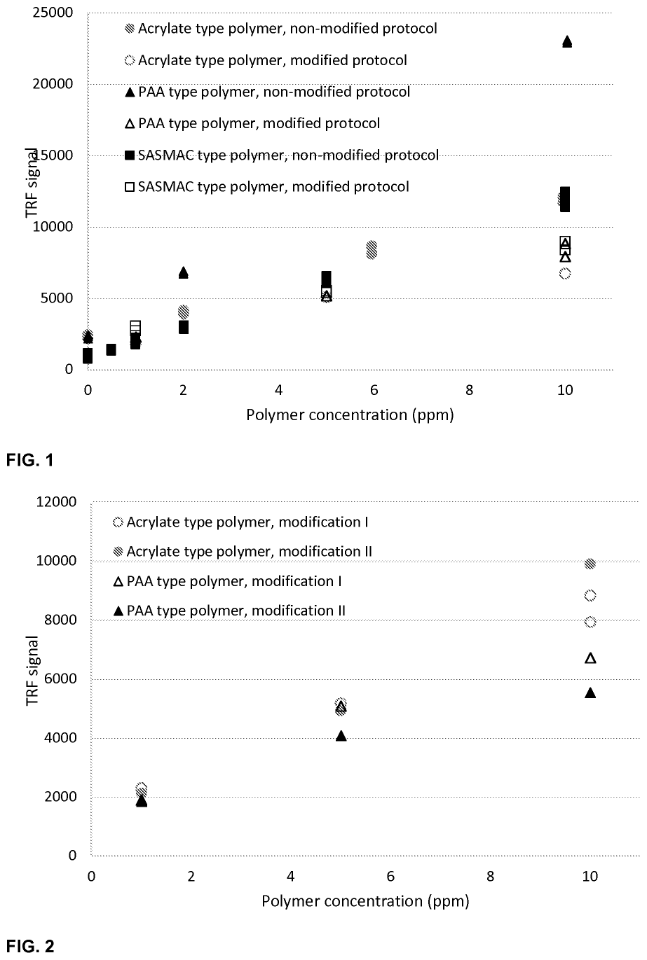

The signal is measured from non-modified polymers and from polymers modified with the method of the present invention. In non-modified protocol, the pH is adjusted to 6.8 using HEPES buffer. In modified protocol, the pH is ˜5 in the reaction. No buffer is used in the modified protocol. As can be seen from FIG. 1 the signals of the modified polymers are at same level compared to signals of non-modified polymers.

Table 1 presents comparison of the TRF signals of lanthanide-polymer chelates measured with non-modified protocol and modified protocol. In non-modified protocol, the pH is adjusted to 6.8 using HEPES buffer. In modified protocol (modification I), the pH is ˜5 in the reaction. No buffer is used in the modified protocol. Max difference of the TRF signals−maximum difference of the polymer TRF signal as compared to other polymers measured (with same concentration range). It can be observed that the differences between the signals are ˜35-130% for non-modified protocol, whereas the differences in the TRF signals are ˜5-50% for modification (mostly, below 35%).

TABLE 1

| | | | | Max difference TRF | |
|---|---|---|---|---|---|
| | | TRF signal | | of the signals | |
| | Concen- | Non- | | Non- | |
| | tration | modified | Modified | modified | Modified |
| Polymer | (ppm) | protocol | protocol | protocol | protocol |
| polyacrylic | 1 | 4520* | 2290 | 130% | 20% |
| acid, pH of | 5 | 12720* | 5170 | 100% | 5% |
| product 3.90 | 10 | 23000 | 8370 | 100% | 25% |
| sodium | 1 | 3260* | 1890 | 70% | 35% |
| polyacrylate, | 5 | 8380 | 5070 | 35% | 5% |
| pH of | 10 | 11880 | 6710 | 50% | 20% |
| product 6.66 | | | | | |
| SASMAC | 1 | 1990 | 2870 | 55% | 50% |
| polymer | 5 | 6380 | 5470 | 50% | 10% |
| | 10 | 11960 | 8670 | 50% | 30% |

FIG. 2 illustrates Europium time resolved fluorescence signal with 0-10 ppm polymeric scale inhibitors. Used polymers are polyacrylic acid (PAA type polymer), pH of product 3.90; and sodium polyacrylate (acrylate type polymer), pH of product 6.66. In modification I, the pH of the sample is adjusted to ˜5. No buffer is used in the modified protocol. In modification II, the pH of the sample is adjusted to ˜5 prior to measurement and HEPES buffer is used. The pH of the measurement solution is ˜6. The concentration of Eu is 11.4 μM in all the tests.

1. A method for determining total concentration of polyelectrolyte or phosphonate in a sample comprising two or more polyelectrolytes and/or phosphonates, the method comprising:
optionally diluting and/or purifying the sample;
acidifying the sample to pH less than 7;
admixing the acidified sample with a reagent comprising lanthanide(lll);
allowing the polyelectrolyte or phosphonate in the sample to interact with the reagent comprising the lanthanide(lll) ion;
exciting the sample at an excitation wavelength and detecting a sample signal deriving from the lanthanide(lll) ion at a signal wavelength by using time-resolved fluorescence; and
determining the total concentration of the polyelectrolyte or phosphonate in the sample by using the detected sample signal.

2. The method according to claim 1, wherein additionally a buffer is admixed with the sample, preferably non-chelating buffer, more preferably zwitterionic Good's buffer, even more preferably HEPES or tris-bis propane.

3. The method according to claim 1, wherein the pH of the sample is adjusted to less than 6, preferably to range 4-6.

4. The method according to claim 1, wherein the sample is acidified with an acid selected from the group consisting of HCl, HNO₃, H₂SO₄ and H₃PO₄.

5. The method according to claim 1, wherein concentration of the lanthanide(lll) ion in the measurement mixture is in a range of 1-100 μM, preferably 1-20 μM.

6. The method according to claim 1, wherein concentration of the polyelectrolyte or phosphonate in the measurement mixture is in a range of 0.5-50 ppm, preferably 5-20 ppm.

7. The method according to claim 1, wherein the lanthanide(lll) ion is selected from europium, terbium, samarium or dysprosium ions, preferably europium or terbium ions.

8. The method according to claim 1, wherein the lanthanide(lll) is a lanthanide(lll) salt, preferably halogenide or oxyanion, more preferably hydrated halogenides or nitrates, most preferably hydrated chloride.

9. The method according to claim 1, wherein the sample is purified by using a purification method selected from the group consisting of centrifugation, size exclusion chromatography, cleaning with solid-phase extraction (SPE) cartridges, dialysis techniques, extraction methods for removing hydrocarbons, filtration, microfiltration, ultrafiltration, nanofiltration, membrane centrifugation and any combinations thereof.

10. The method according to claim 1, wherein the polyelectrolyte contains one or more groups selected from, carboxylic acid/carboxylate, amide, phosphonate, amine, or any combination thereof.

11. The method according to claim 1, wherein at least one of the polyelectrolytes or phosphonates is tagged, preferably with fluorescent tag with different excitation and emission wavelengths as compared to the used lanthanide(III).

12. The method according to claim 11, wherein after the determination of the total concentration of the polyelectrolyte or phosphonate in the sample, the sample is
excited at excitation wavelength specific for the tag and detecting a sample signal deriving from the lanthanide(lll) ion at a signal wavelength specific for the tag by using time-resolved fluorescence; and
determining the concentration of the at least one tagged polyelectrolyte or phosphonate in the sample by using the detected sample signal.

13. Use of the method according to claim 1 for determining total concentration of polyelectrolyte or phosphonate in a sample.

14. The use according to claim 13, wherein the sample originates from water treatment, paper making processes, pharmaceutical industry, well drilling, mineral processing, enhanced oil recovery, an oilfield or an oil well or from an oil production process.

15. A device comprising means for performing the method according to claim 1 for determining total concentration of polyelectrolyte or phosphonate in a sample.

16. The method according to claim 4, wherein the acid is HCl.
